(19) United States
(12) Patent Application Publication
(10) Pub. No.: US 2021/0341467 A1
(43) Pub. Date: Nov. 4, 2021

(54) PORTABLE DEVICES AND METHODS FOR DETECTING AND IDENTIFYING COMPOUNDS IN SALIVA

(71) Applicant: Pelican Diagnostics Inc., Burlington (CA)

(72) Inventor: Raj Reddy, Burlington (CA)

(21) Appl. No.: 16/933,525

(22) Filed: Jul. 20, 2020

Related U.S. Application Data
(60) Provisional application No. 63/019,985, filed on May 4, 2020.

(51) Int. Cl. G01N 33/543; G01N 33/577; B01L 3/00; C12M 3/00; C12M 1/36

(52) U.S. Cl. CPC G01N 33/54306; G01N 33/577; B01L 3/5029; B01L 2300/0681; C12M 41/48; G01N 33/5438; C12M 23/54

(57) ABSTRACT

This disclosure relates to portable devices for SARS-COV-2 Antigen (SC2A), a biomarker for the detection of Coronavirus disease 2019 (COVID19). Diagnosis of viral infections such as SARS-COV-2 can be obtained in the early stages of a disease by detection of viral antigens (e.g., SC2A) directly in the clinical specimen.

### CROSS-REFERENCE TO RELATED APPLICATIONS

This application claims the benefit of U.S. Provisional Application No. 63/019,985, filed May 4, 2020, the entire contents of which are incorporated herein by reference in their entirety.

### TECHNICAL FIELD

This disclosure relates to SARS-COV-2 Antigen (SC2A), a biomarker for the detection of Coronavirus disease 2019 (COVID19), and the use of a portable device. Diagnosis of viral infections, such as SARS-COV-2, can be obtained in the early stages of a disease by the detection of a viral antigen (e.g., SC2A) and at later stages by detecting antibodies directly in a clinical specimen. Developing an electrical biosensor for antigen detection offers a great possibility to create a low-cost and highly sensitive sensor as a point of care method to detect and quantify one or more compounds in saliva, blood, urine or other bodily liquids in real-time. This design offers the advantage of a label free design. In one embodiment we will focus on the saliva antigen to COVID19. In other embodiments we will detect antibodies in the saliva or blood.

### BACKGROUND

Approximately 1400-2000 proteins have been identified in saliva, including various antibody compounds. The number of proteins found in saliva demonstrates its diversity for disease detection, and each of these proteins can be used as a simple tool to assess at least toxicity, infectiousness, immunology, and hormonal levels. Several independent studies have shown that a saliva antigen is directly correlated with COVID19 in patients and therefore can be a useful antigen-based biomarker.

A point of care sensor would also drastically decrease the need for a sample to be transported to a main laboratory and would be helpful in building new economical outcomes in health care management. Previously developed biosensor chips utilized an invasive approach and required serum/whole blood as a sample to detect the level of a compound. The most common techniques previously reported to detect antibodies at a point of care level include surface plasmon resonance, surface enhanced raman scattering, fluorescent assay, electrochemical impedance spectroscopy, cyclic voltammetry, electrochemiluminescent, amperometric, capacitance and photo-electrochemical. Numerous studies have published point of care detection of antibody compounds utilizing serum/whole blood samples. In comparison to blood, saliva sample collection is less invasive and therefore requires lower cost making the detection of antibodies truly point of care. Saliva samples can easily be collected at lower cost putting a lesser burden on patients as compared to the collection of blood. The range of SC2A in a saliva sample is in 0.01-1.74 ng/mL as measured using micro-particle enzyme immunoassay technology. Investigators have studied the saliva in low-serum antigen concentration groups and results were reported using RT-PCR. A point of care biosensor for detection of an antigen (e.g., SC2A) in human saliva is not known (although laboratory-based techniques are available for saliva antigen detection) and a point of care biosensor for the detection of an antibody in humans is known for other conditions such as HIV. The success of this research will help to make antibody detection point of care in a true sense.

Accordingly, there exists a need for automated portable devices and methods for directly detecting antigen compounds in virus infected patient saliva, and which provide analytical results in real time with concurrent reporting to remote users, such as, for example, health care professionals. The area of simple miniaturized devices integrated with biosensors has great significance and prospects of commercialization as a handheld device at low-cost offering great potential for clinical use in a point of care setting. Such devices and methods would also be of significant value in measuring patient compliance with pharmaceutical regimens and/or determining active infection. The same sensor system could also be used for quantitative and qualitative analysis of blood or other bodily fluids samples for antibodies.

### SUMMARY

This disclosure relates to portable devices for detecting antigens and/or antibodies, which may act as biomarkers for the detection of a disease or disorder. For example, a disease or disorder may be detected during the early stages by using a device to detect antigen and/or antibody biomarkers in a body fluid sample (e.g., blood or saliva) of a subject. In some embodiments, a biosensor may be used to detect the antigen and/or antibody biomarkers in the sample. In some aspects, the disclosure relates to portable devices for detecting SARS-COV-2 Antigen (SC2A), a biomarker for the detection of COVID19 disease (C19). Diagnosis of viral infections such as SARS-COV-2 can be obtained in the early stages of a disease by detection of viral antigens (e.g., SC2A) directly in the clinical specimen. Developing an electrical biosensor for antigen detection offers a great possibility to create a low-cost and highly sensitive sensor as a point-of care test to detect and quantify the compounds in a body fluid sample (e.g., saliva or blood) in real-time. A biosensor may be integrated with a portable device to provide electrical connection and to wirelessly transmit/receive electrical signals. This fully integrated proposed handheld device successfully exhibits a wide compound lowest detection range with a high sensitivity.

Disclosed herein are portable devices and methods for diagnosing a virus (e.g., COVID-19) by detecting and identifying an antigen in saliva, blood or other bodily fluids and/or detecting antibodies (e.g., COVID19 antibodies) in blood, saliva or other bodily fluids. In some aspects, coatings for sensor substrates with novel sensing elements are provided.

In one embodiment, a portable device for detecting and identifying one or more antigen compounds and/or one or more antibody compounds in a saliva and/or blood sample is provided. In some embodiments, the device includes a biosensor (e.g., a disposable biosensor) connected to an electronic board module (also referred to herein as a sensor module), which collects data that detects and identifies an antigen or antibody compound in the saliva of a subject. The sensor module may be disposed in a housing of the portable device. In some embodiments, the portable device includes a communication apparatus connected to the sensor module, which can transmit the data collected by the biosensor to an external processing apparatus. In some embodiments, the device includes a battery disposed in the housing connected to the sensor module and the communication apparatus.

In some embodiments, a processing apparatus is electrically or wirelessly connected to the communication apparatus. The processing apparatus may analyze data transmitted by the communication apparatus to detect and identify the one or more compounds in the saliva or blood of the subject.

In some embodiments, the portable device further comprises an amplifier connected to the sensor module. The amplifier may amplify data collected by the sensor module.

In some embodiments, the antigen compounds are detected in real time. In some embodiments, the antigen compounds are immobilized with at least one nanoparticle. The immobilized antigen compounds include some marker compounds. In some embodiments, the identified and detected compound is a marker compound. A marker compound may be a viral antigen (e.g., SC2A, IgG, anti-M2, etc.). In some embodiments, the marker compound is functionalized or immobilized with graphene nanoplates and block co-polymer inorganic. In some embodiments, the antigen compounds are viral antigens (e.g., SC2A, IgG, anti-M2, etc.). In some embodiments, the presence of a viral antigen is an indicator or a marker that a subject is suffering from an infection, e.g., a SARS-CoV-2 infection.

Also disclosed herein is a coating for a sensor substrate. In one embodiment, the coating may include a nanoparticle (e.g., carbon-based nanomaterials), one or more marking compounds embedded in the nanoparticle, and a polymer matrix.

Also disclosed herein is a sensor. In one embodiment, the sensor may include one or more antibodies on a substrate form, and a coating covering. The coating covering may include a functionalized inorganic metallic oxide nanoparticle and a polymer matrix.

In another embodiment, the sensor may include an interdigital electrode, which includes multi-walled carbon nanotubes that are attached to one or more antibody molecules which the electrodes are disposed.

In other embodiments, the sensor may include an interdigital electrode, which includes magnetic nanoparticles and antibody functionalized gold nanoparticles which the electrodes are disposed.

In some embodiments, the sensor may include an interdigital electrode, which includes two different shaped antibody functionalized gold nanoparticles which the electrodes are disposed.

Disclosed herein is a sensor comprising gold coated interdigital electrodes, wherein the interdigital electrode comprises a nanocomposite of graphene nanoplatelet with deblock-co-polymer, which includes one or more immobilizing antigen molecules; and a support on which the electrodes are disposed.

Also disclosed herein are methods for detecting and identifying antigen or antibody compounds from the saliva or blood of a subject. The methods may comprise collecting and, after chemical treatment of the saliva or blood collected from a subject, analyzing the saliva or blood in a device, wherein a housing of the device includes a sensor module, a communication apparatus, and a battery. The method may further include collecting data about the presence and identity of the antigen compounds with the sensor module and communicating the data via the communication apparatus to a processing apparatus. The method may further include processing the communicated data to detect and identify one or more antigen or antibody compounds.

### BRIEF DESCRIPTION OF THE DRAWINGS

The patent or application file contains at least one drawing executed in color. Copies of this patent or patent application publication with color drawings will be provided by the Office upon request and payment of the necessary fee.

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates a flow chart of laboratory diagnostics of a virus.

FIG. 2 illustrates an example of a portable device which identifies and detects an antigen and/or antibody in the saliva of a subject.

FIG. 3 illustrates a sensor which includes an immobilized antibody with a sensing coating element which may be used in the sensor module of a portable device which identifies and detects an antigen and/or antibody compound in the saliva of a subject.

FIGS. 4A-4D illustrate a real time analysis of human saliva samples.

### DETAILED DESCRIPTION

Disclosed herein are portable devices and methods for detecting and identifying antigen and/or antibody compounds in saliva of a subject. Also disclosed herein are coatings for sensor substrates, as well as novel sensors.

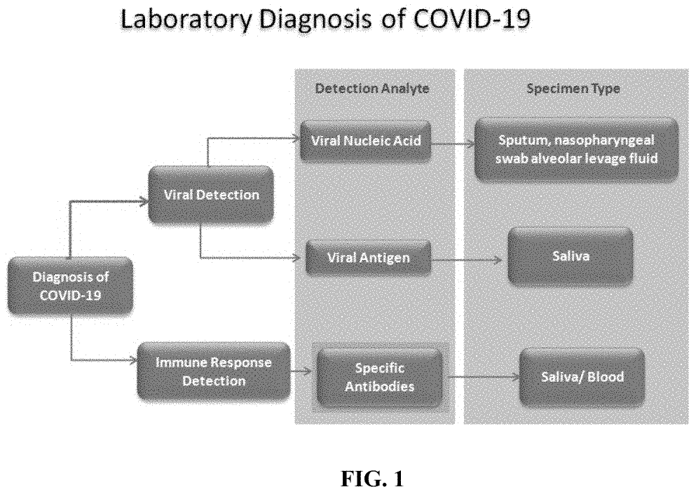

Referring to FIG. 1, which illustrates a flow chart of laboratory diagnosis of COVID-19 by detecting an antigen from a saliva sample. Viral isolation and a number of methods for the detection of viral antigens, nucleic acids, and antibodies are the core repertoire of techniques used for the laboratory diagnosis of viral infections, although some other techniques are also occasionally used. Viral isolation by means of cell culture is generally performed in designated virology laboratories. Other methods of detection may be performed in virology laboratories as well but may also be performed in diverse laboratory sections such as general microbiology, serology, blood bank, clinical chemistry, pathology, or molecular virology. The trend for viral diagnostic testing to be done outside of traditional virology laboratories is likely to accelerate as rapid diagnostic techniques based on immunologic and nucleic acid methodologies increasingly replace viral culture. Serum and whole blood samples may be required. Throat swabs, urine specimens, and CSF specimens may also be useful, depending upon the infection. Public health laboratories may be consulted regarding the choice of specimen, specimen collection, and transport.

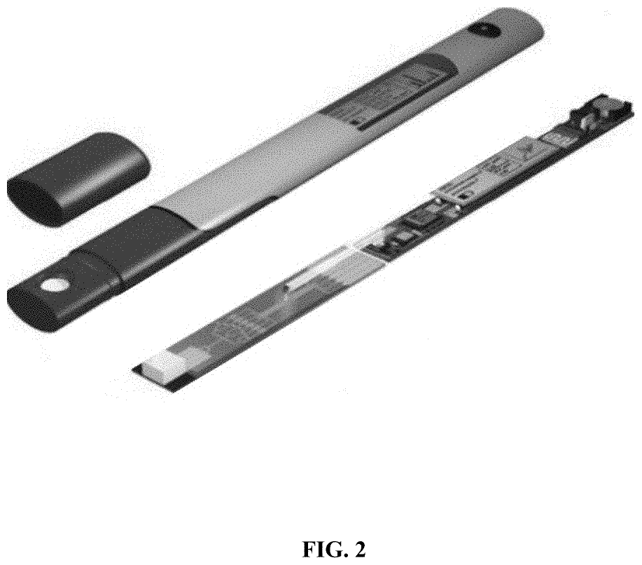

In some embodiments, a portable device, such as the one illustrated in FIG. 2, collects data with a sensor module about the presence and the identity of compounds in a sample (e.g., saliva) from a subject. In some embodiments, the sensor module converts collected data to a signal (e.g., provides significantly high electrical conductivity, thermal conductivity, optical etc.), and transmits the data via a communication apparatus to a processing apparatus which analyzes the data to provide information about the presence and identity of compounds in the saliva of the subject. In some embodiments, the processing apparatus processes the received signals to provide information about the presence and identity of the compound (e.g., antibody) in the saliva of a subject. In some aspects, the processing apparatus may transmit the presence and identity of the one or more compounds to a display.

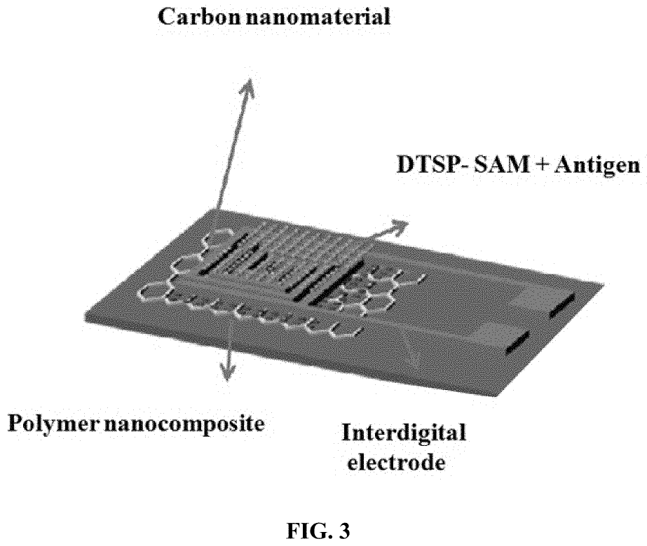

FIG. 3. illustrates a schematic description of an Inter-Digitated Electrode-based biosensor, although other embodiments may use other materials, such as paper. In some embodiments, a sensor substrate antibody is conjugated using 3,3′-Dithiodipropionic acid (N-hydroxysuccinimide ester) (DTSP) —self-assembled-monolayer (SAM) to gold electrodes, which are deposited on top of one or more layers of composite containing polymers and carbon nanomaterials. In some embodiments, sensors may include biomolecule functionalized gold nanoparticles, composites of magnetic nanoparticles with antigen immobilized gold nanoparticles, composites of different size surface modified gold nanoparticles, coordination polymers with carbon black composites, copolymer-carbon black composites, conducting inorganic or organic materials, immobilized metal organic frameworks, or nanocomposites of graphene nanoplatelet with diblock-co-polymer or biological materials, such as enzymes, antibodies, nucleic acids, etc. to recognize the presence and identity of compounds in the saliva of the subject. In certain embodiments, the sensors described herein convert detection of the presence of antigen compounds in the saliva of the subject to a signal (e.g., electrical, optical, thermal, etc.), which is transmitted to a processing apparatus for analysis, identification, and quantification.

In some embodiments, a simple electrochemical sensor for identifying and detecting antigen compounds (e.g., SC2A, IgG, anti-M2, etc.) in saliva of a subject comprises an electrode (e.g., an interdigital electrode). In some embodiments, the electrode is copper base and gold coated. Electrodes which may be used in the sensors described herein include, but are not limited to, biofunctionalized gold nanoparticles, antigen immobilized gold nanoparticles, copolymer-carbon black composites, block copolymer carbon black nanocomposites, graphene nanoplate with diblock copolymer nanocomposites, etc. The pure graphitic composition on a sensor substrate provides significantly high electrical and thermal conductivity while diblock co polymer makes an amphiphilic bridge between graphene units.

Graphene is an allotrope of carbon, in the form of a single layer of atoms in a two-dimensional hexagonal lattice in which one atom forms each vertex. Graphene may have many potential applications in a wide variety of industries due to its many extraordinary properties coupled with nanometer-scale size, and therefore is accordingly well know and readily available. In some embodiments, graphene nanocomposites are used as a sensing element.

In some embodiments, a base material of a multilayer biosensor includes a graphene-polymer nanocomposite. The graphene-polymer composite may be coated on to make it suitable for absorbing the fluid component of a saliva sample. Electrochemical immunosensing is based on the principle of measuring the changes in electrical properties of a conductive material due to the adsorption of an analyte on the surface functionalized with antibodies.

In some embodiments, square rings based on gold material were fabricated as innovative design electrodes for antigen detection to achieve better accuracy and sensitivity. In some aspects, the small electrode setup is a key step to the fabrication of the biosensor. This new design based on square rings increased the sensitivity of detection and introduced a dynamic sensing mechanism with an actual sensing area of about 1×1 mm². In some embodiments, the inter electrode spacing between square electrodes is about 45 μm along with individual electrode width of 45 μm. In some embodiments, the total area covered by the electrodes is about 495 μm² (11×45 μm), total interspacing between electrodes is about 450 μm² (45×10), and total covered sensing area is approximately 1 mm².

In some embodiments a smaller electrode size is used. The smaller electrode size may provide only enough space to conjugate DTSP-SAM/antigen for complete attachment to a gold electrode thereby leaving smaller areas of no antibody attachment, which may create non-specific antigen binding. In some aspects, reducing the size and thickness of gold electrodes decreases the electrical field on the gold surface which finally limits the detection procedure.

In some embodiments, electrochemical impedance spectroscopy and cyclic voltammetry are the standard techniques utilized to detect a bioanalyte on the sensor surface. A low-cost, easy-to-use, simple and re-configurable miniaturized electronics sensor module is described herein to quantify the biosensor before and after treating a fluid (e.g., saliva) sample with immobilized surface antibodies.

In some embodiments, DC voltage of about 5V was applied directly to the microcontroller embedded in a sensor module which then transferred signals to the communication device and display device. In some embodiments, electrical signals monitor on a trained data analytics machine a learning algorithm in real-time fashion. Readings may record in terms of electrical resistance. In some aspects, electrical resistance can easily be displayed into current and voltage gain if required by reconfiguring the program for a microcontroller.

In certain aspects, a battery is a lithium ion battery. Lithium ion batteries are conventional and may be available from many commercial sources (e.g., PromasterDMW-BCM13E), such as rechargeable battery packs. Many batteries are known in the art and may be used in the portable devices described herein.

In some embodiments, the processing apparatus is a conventional general-purpose computer, which includes a display device and a communication interface which allows reception and transmittal of information from other devices and systems via any communication interface. In some aspects, the processing module typically detects and identifies the antigen compounds in the saliva of the subject by processing the data received from the sensor module with results sent to the display device. Any general purpose computer known in the art which has sufficient processing power to analyze data provided by the sensor module may be used in conjunction with the portable devices described herein.

In some embodiments, data from sensors in the sensor module is analyzed using pattern and recognition systems such as, for example, artificial neural networks, which include, for example, multi-layer perception, generalized regression neural network, fuzzy inference systems, etc. and statistical methods such as principal component analysis, partial least squares, multiples linear regression, etc. In some aspects, artificial neural networks are data processing architectures that use interconnected nodes (i.e., neurons) to map complex input patterns with a complex output pattern. Importantly, neural networks can learn from using various input output training sets.

In some embodiments, the one or more compounds which are detected and identified using the portable devices are detected and identified directly. For example, a certain antigen may be detected by the portable device. Antigens which may be directly identified and detected include, but are not limited to, SC2A, Capsid antigen, HCV antigen, E antigen, HBsAg, antiHSV-2, Anti-H1N1, HA gene, particles, L1 gene, p24 antigen, Immunoglobulin G (IgG), PSA-antigen, corstisol, ImG, pp65 antigen, or anti-CMV.

In some embodiments, record impedance based electrical signals for antigen detection with a low detection of about 40 fg/mL, which is lower than 13 pg/mL, 200 pg/mL, 15 pg/mL, 2.3 pg/mL, 2.7 pg/mL and 1 pg/mL for electrochemical sensors, 91 pg/mL and 0.29 ng/mL for SPR sensors, 27 pg/mL, 0.2 ng/mL, 40 pg/mL and 0.3 pg/mL for fluorescent assay, 0.9 ng/mL for colorimetric, 0.11 pg/mL for SERS, 0.1 ng/mL for electrical immunosensor, 0.6 ng/mL for sandwich-type ELISA impedimetric immunosensor, 0.29 pg/mL for ECL immunosensor and 2.6 pg/mL for PEC immunosensor.

In some embodiments, a coating for a sensor substrate is provided. In one embodiment, the coating may include carbon-based nanomaterials, a marker compound embedded in the carbon-based nanomaterials, and a polymer matrix.

In some embodiments, the marker compound is an antigen detector in saliva by using an immobilized prostate specific antigen with a nanocomposite of graphene nanoplatelet with diblock-co-polymer or similar co-polymer consisting of two or more monomers and gold electrodes.

In some embodiments, a marker compound exhibited a wide range of antigen detection, for example, 50 fg/mL-10 ng/mL by utilizing a nanocomposite of SnS₂@mpg-C₃N₄ with high specific surface area and large pore volume. In one aspect, to further enhance the current response, ascorbic acid (an electron donor) was used which played a significant role to the immunosensor measurement with LoD 21 fg/mL.

In some embodiments, an antibody was covalently linked on gold electrode via DTSP/SAM conjugation chemistry. In some aspects, the thickness of the coating is between about 0.1 microns and about 50 microns.

Those of skill in the art will appreciate that combinations of nanoparticles with different marker compounds at varying concentrations can be used to create a vast library of unique coatings.

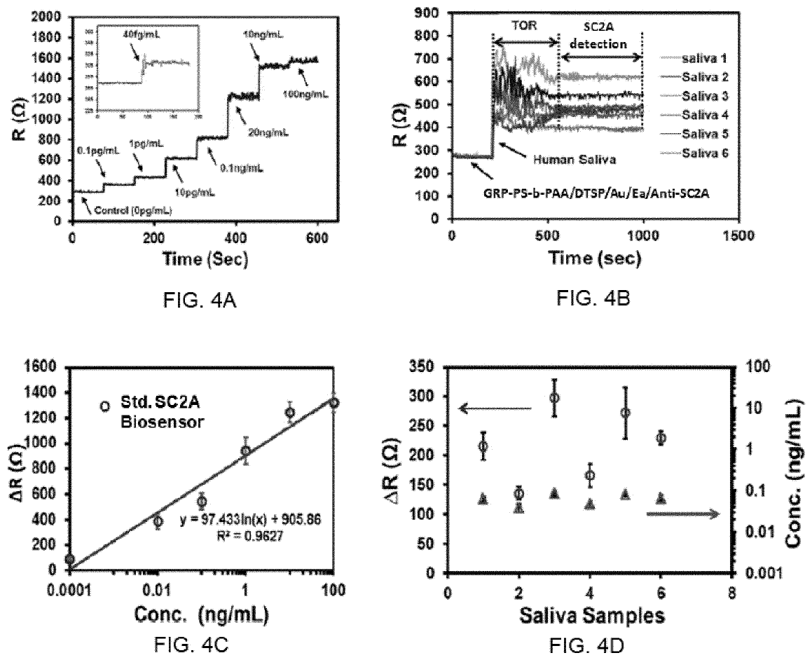

FIG. 4 provides an example illustration demonstrating standard antibody aqueous solution and human saliva samples tested with an electrical biosensor. In FIG. 4A the change in electrical resistance increased with increasing SC2A concentration (40 fg/mL-100 ng/mL). In FIG. 4B the calibrated range for SC2A solution with RoD of 0.1 pg/mL-100 ng/mL (R2=0.963) and LoD of 40 fg/mL. In FIG. 4C a real time analysis is provided of human saliva samples (#1, 2, 3, 4, 5 and 6). TOR indicates time of response to achieve the stable signal for SC2A presence in saliva samples. In FIG. 4D the change in resistance (ΔR) corresponds to SC2A concentration 0.063, 0.039, 0.086, 0.048, 0.079 and 0.067 ng/mL in respective samples. ΔR indicates the difference in resistance of the biosensor chip before and after testing saliva samples. Error bars represent the relative SD for n=4.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited.

1. A portable device for detecting and identifying antigen compounds in the saliva or antibodies in the blood of a subject comprising:
a. a biosensor connected to a device;
b. a sensor module disposed in a housing of the device, wherein the sensor module collects data, and thereby detects and identifies one or more antigen compounds and/or one or more antibody compounds in a saliva or blood sample;
c. a communication apparatus disposed to the sensor module, wherein the communication apparatus electrically transmits the collected data; and
d. a battery disposed to the communication apparatus and the sensor module.

2. The portable device of claim 1, further comprising a processing apparatus electronically or wirelessly connected to the communication apparatus, wherein the processing apparatus processes the data transmitted by the communication apparatus to detect and identify the compound.

3. The portable device of claim 2, wherein the processing apparatus transmits the identity of the compound detected in a sample from the processing apparatus to a display.

4. The device of claim 1, further comprising an amplifier connected to the sensor module, wherein the amplifier amplifies data collected by the sensor module.

5. The device of claim 1, wherein an antigen compound or antibody compound is detected in real time.

6. The device of claim 1, wherein an antigen compound is immobilized with one or more nanoparticles, wherein the one or more nanoparticles include one or more marker compounds.

7. The device of claim 6, wherein the antigen compound identified and detected is a marker compound.

8. The device of claim 7, wherein the marker compound is functionalized or immobilized with graphene nanoplates and block co-polymer inorganic.

9. The device of claim 5, wherein the antigen compound is a viral antigen.

10. The device of claim 9, wherein the viral antigen is selected from the group consisting of SARS-COV-2 Antigen (SC2A), Immunoglobulin G (IgG), and anti-mitochondrial M2 (anti-M2).

11. The device of claim 9, wherein the viral antigen is SC2A.

12. A sensor comprising:
a. a gold coated interdigital electrode, wherein the interdigital electrode comprises a nanocomposite of graphene nanoplatelet with diblock-co-polymer, wherein the nanocomposite includes one or more immobilized antigen molecules; and
b. a support on which the electrodes are disposed.

13. A method for detecting and identifying antigen compounds from a fluid sample of a subject comprising:
a. collecting and chemically treating a fluid sample of a subject;
b. collecting the sample in a connected device, wherein the device comprises a housing including at least a sensor module having at least one sensor, a communication apparatus, and a battery, wherein the at least one sensor comprises one or more electrodes and at least one antibody bound to the one or more electrodes via 3,3′-Dithiodipropionic acid (N-hydroxysuccinimide ester) (DTSP) —self-assembled-monolayer (SAM);
c. collecting data about the presence and identity of one or more antigen compounds with the sensor module, wherein a first antigen compound binds to the at least one antibody bound to the one or more electrodes;
d. communicating collected data via the communication apparatus to a processing apparatus; and
e. processing the communicated data to detect and identify the one or more antigen compounds, wherein the first antigen compound is identified as SARS-COV-2 antigen (SC2A).

14. The method of claim 13, wherein a second antigen compound detected and identified is a viral antigen that is not SC2A.

15. The method of claim 14, wherein the viral antigen is selected from the group consisting of IgG and anti-M2.

16. (canceled)

17. The method of claim 13, wherein the SC2A antigen is an indicator of the subject suffering from a SARS-COV-2 infection.

18. The method of claim 13, wherein the one or more antigen compounds are detected at a concentration of about 40 fg/mL to 100 ng/mL.

19. The method of claim 13, wherein the one or more antigen compounds are detected at a concentration of about 50 fg/mL to 10 ng/mL.

20. The method of claim 13, wherein the collected data is converted to an electrical signal prior to communication to the processing apparatus.
